(19) United States
(12) Patent Application Publication
(10) Pub. No.: US 2005/0214235 A1
(43) Pub. Date: Sep. 29, 2005

(54) Methods of treating hair

(76) Inventors: Guy LaTorre, Gainesville, FL (US); Kevin McKenzie, High Springs, FL (US)

Correspondence Address: HUTCHISON & MASON PLLC, PO BOX 31686, RALEIGH, NC, 27612, US

(21) Appl. No.: 10/506,522

(22) PCT Filed: Mar. 7, 2003
(86) PCT No.: PCT/US03/07087

§ 371 Date: May 17, 2005

Related U.S. Application Data
(60) Provisional application No. 60/362,359, filed on Mar. 7, 2002.

(51) Int. Cl. A61K 7/06

(52) U.S. Cl. 424/70.1

(57) ABSTRACT

Methods for treating hair are disclosed. Formulations for use in the methods for treating hair comprising bioactive glass compositions and a suitable carrier are also disclosed. The methods disclosed involve application of hair treatment formulations to hair which formulations include bioactive glass compositions which may comprise non-interlinked particles of bioactive glass or bioactive glass extract, and a carrier, and/or optional components useful in hair treatment formulations.

### BACKGROUND OF THE INVENTION

1. Field of the Invention

The present invention relates to compositions and methods of use in the treatment of hair.

2. Background

Hair is composed of a class of natural fibrous proteins called keratin. This is the same kind of protein that makes up the nails and the outer layer of skin. The softer keratins are components of the external layers of skin, wool, hair and feathers, while the harder types predominate in nails, claws and hoofs. A cross section taken through a terminal hair shaft reveals two distinct components, the cuticle and the cortex. The cuticle is the outermost surface of the hair shaft and is composed of a very hard keratinous substance, while the inner bulk of the hair is composed of a more fibrous keratin and is known as the cortex. A third element, the medulla, is composed of a softer keratin-rich material and its occurrence in human hair appears to be variable, and is typically found in large, thick hairs.

Chemically, keratin is an insoluble proteinaceous complex composed of high molecular weight polypeptide chains. These polypeptide chains are composed of a series of amino acids joined head-to-tail by peptide linkages, the actual properties of the polypeptide being determined by the number, type and order of amino acids in the chain. Typically, keratins are insoluble in organic solvents but do absorb and hold water.

The cuticle of hair generally consists of flattened platelets of amorphous keratin, wrapped around the hair shaft in several layers, each layer overlapping the adjacent one progressing from the root of the hair to its tip. The condition of the cuticle is responsible for the outward appearance of the hair and dictates the attributes of feel and shine. In virgin hair, the cuticle platelets lay flat against each other and are firmly adhered to the cortex, giving rise to a very smooth feeling surface with a high degree of shine. If the hair is subjected to environmental or physical damage, the cuticle platelets can become chipped, raised or even detached completely, exposing the cortex underneath. This gives rise to hair in poor condition, which feels rough to the touch and is dull in appearance.

The most frequent cause of hair damage is mechanical abrasion, typically combing or brushing, often in combination with the use of hair treatment products such as perming lotions, colorants or even shampoos. The extent of physical damage depends, to a large extent, on the coefficient of friction between the comb or brush and the surface of the hair itself. This value rises dramatically if the hair is wet with the consequence that far more damage is normally inflicted to the hair if it is combed or brushed when wet, rather than when dry. The cuticle of the hair is also responsible for the water repellent properties of the hair. If the cuticle is badly damaged or removed completely, water rapidly penetrates the cortex which subsequently swells, making the hair more prone to further mechanical damage.

Various compositions and methods have been used to prevent or treat damaged hair. U.S. Pat. No. 3,864,475, for example, relates to a method of treating human hair to enhance softness and improve the general appearance by intimately contacting the hair with an aqueous treating composition containing water and a catalytically effective amount of a catalyst. The catalyst may be prepared by admixing a water soluble alkali metal silicate with an aqueous medium containing a dissolved substance which is source of calcium ion and a dissolved substance which is a source of magnesium ion to produce a finely divided or colloidal suspension of the reaction product. The colloidal suspension is then agitated sufficiently in the presence of a micelle-forming surfactant to form catalyst-containing micellees.

U.S. Pat. No. 3,958,581 relates to the treatment of degraded hair by use of cosmetic compositions containing at least one cationic polymer and at least one non-toxic salt of a divalent metal.

U.S. Pat. No. 5,635,168 teaches compositions and methods for treating hair using an aqueous composition of at least 1% by weight total of one or more polyvalent metal compounds; a sulfur-containing material that has an average molecular weight of 10,000 or less that can form disulfide bonds involving the keratin of the hair, and whose sulfur content is at least about 1% by weight, and optionally, an acid.

There is a continuing need in the art for a hair treatment and composition which builds body in hair end provides smooth, silky hair. The methods and compositions of the present invention provide such compositions and treatment.

### BRIEF SUMMARY OF THE INVENTION

Methods for treating hair are disclosed. These methods include treating hair with bioactive glass compositions which comprises particulate bioactive glass or bioactive glass extracts. The bioactive glass compositions may further comprise a carrier and/or optional components useful in hair treatment formulations.

In one aspect of the invention, the method comprises applying an effective hair-enhancing amount of a bioactive glass composition to the hair for a sufficient amount of time such that the hair is coated with the bioactive glass composition. The coating comprises a film comprising silicon, calcium, and/or phosphorus ions which may interact with the hair. Additionally, sodium or other ions released from the bioactive glass composition may be present. The hair after application of the bioactive glass composition is smooth and silky with increased body.

In another aspect of the invention, the method comprises applying an effective hair-enhancing amount of bioactive glass extract(s) (solutions with ions and substantially no particles) to the hair for a sufficient amount of time such that the hair is coated with a precipitated film or coating comprising silicon, phosphorous and calcium. Additionally, sodium or other ions released from the bioactive glass extracts may be present.

In another aspect of the invention, the method comprises applying a shampoo or conditioner to hair, the shampoo or conditioner comprising an effective hair-enhancing amount of a bioactive glass composition which may comprise non-interlinked particles of bioactive glass or bioactive glass extract.

### BRIEF DESCRIPTION OF THE DRAWINGS

FIG. 1 is a scanning electron microscopy (SEM) micrograph at a magnification of 500× showing film formation on hair after treatment with the bioactive glass and tris buffer formulation of Group #1 described in the Example.

FIG. 2 is a scanning electron microscopy (SEM) micrograph at a magnification of 4500× showing film formation on hair after treatment with the bioactive glass and tris buffer formulation of Group #1 described in the Example.

FIG. 3 is an Energy Dispersive Spectrometer (EDS) spectrum showing the presence of silicon, calcium and phosphorus on hair after treatment with the bioactive glass and tris buffer formulation of Group #1 described in the Example.

FIG. 4 is a Fourier Transform Infrared Spectroscopy (FTIR) spectrum which illustrates that the calcium and phosphorus in the film are in the form of crystalline hydroxyapatite.

FIG. 5 is a scanning electron microscopy (SEM) micrograph at a magnification of 500× showing film formation on hair after treatment with the bioactive glass extract formulation of Group #2 described in the Example.

FIG. 6 is a scanning electron microscopy (SEM) micrograph at a magnification of 4500× showing film formation on hair after treatment with the bioactive glass extract formulation of Group #2 described in the Example.

FIG. 7 is an Energy Dispersive Spectrometer (EDS) spectrum showing the presence of silicon, calcium and phosphorus on hair after treatment with the bioactive glass extract formulation of Group #2 described in the Example.

FIG. 8 is a scanning electron microscopy (SEM) micrograph at a magnification of 500× showing lack of film formation on hair after treatment with the tris buffer control for Groups #1 and #2 described in the Example.

FIG. 9 is a scanning electron microscopy (SEM) micrograph at a magnification of 4500× showing lack of film formation on hair after treatment with the tris buffer control for Groups #1 and #2 described in the Example.

FIG. 10 is an Energy Dispersive Spectrometer (EDS) spectrum showing the absence of silicon, calcium and phosphorus on hair after treatment with the tris buffer control for Groups #1 and #2 described in the Example.

FIG. 11 is a scanning electron microscopy (SEM) micrograph at a magnification of 500× showing film formation on hair after treatment with the bioactive glass, tris buffer and shampoo formulation of Group #3 described in the Example.

FIG. 12 is a scanning electron microscopy (SEM) micrograph at a magnification of 4500× showing film formation on hair after treatment with the bioactive glass, tris buffer and shampoo formulation of Group #3 described in the Example.

FIG. 13 is an Energy Dispersive Spectrometer (EDS) spectrum showing the presence of silicon, calcium and phosphorus on hair after treatment with the bioactive glass, tris buffer and shampoo formulation of Group #3 described in the Example.

FIG. 14 is a scanning electron microscopy (SEM) micrograph at a magnification of 500× showing film formation on hair after treatment with the bioactive glass extract and shampoo formulation of Group #4 described in the Example.

FIG. 15 is a scanning electron microscopy (SEM) micrograph at a magnification of 4500× showing film formation on hair after treatment with the bioactive glass extract and shampoo formulation of Group #4 described in the Example.

FIG. 16 is an Energy Dispersive Spectrometer (EDS) spectrum showing the presence of silicon, calcium and phosphorus on hair after treatment with the bioactive glass extract and shampoo formulation of Group #4 described in the Example.

FIG. 17 is a scanning electron microscopy (SEM) micrograph at a magnification of 500× showing lack of film formation on hair after treatment with the tris buffer and shampoo control for Groups #3 and #4 described in the Example.

FIG. 18 is a scanning electron microscopy (SEM) micrograph at a magnification of 4500× showing lack of film formation on hair after treatment with the tris buffer and shampoo control for Groups #3 and #4 described in the Example.

FIG. 19 is an Energy Dispersive Spectrometer (EDS) spectrum showing the absence of silicon, calcium and phosphorus on hair after treatment with the tris buffer control for Groups #3 and #4 described in the Example.

### DETAILED DESCRIPTION OF THE INVENTION

The hair treatment methods of the present invention involve application to hair of bioactive glass compositions comprising particulate bioactive glass or bioactive glass extract. The treatment provides a film on the hair which results in hair with greater body, smoothness and silkiness. The hair treated may be hair from or on any mammal such as hair on dogs or cats, but preferably will be human hair.

As used herein, the terms “bioactive glass” or “biologically active glass” mean an inorganic glass material having an oxide of silicon as its major component and which is capable of bonding with growing tissue when reacted with physiological fluids.

Bioactive glasses are well known to those skilled in the art, and are disclosed, for example, in An Introduction to Bioceramics, L. Hench and J. Wilson, etds. World Scientific, New Jersey (1993). The bioactive glass of the present invention typically will contain about 40 to about 86% by weight of silicon dioxide (SiO₂), about 0 to about 35% by weight of sodium oxide (Na₂O), about 4 to about 46% by weight calcium oxide (CaO), and about 1 to about 15% by weight phosphorus oxide (P₂O₅). Preferably, the silicon dioxide is present in an amount of about 40 to about 68% by weight, the sodium oxide is present in an amount of about 5 to about 30% by weight, the calcium oxide is present in an amount of about 10 to about 35% by weight and the phosphorus oxide is present in an amount of about 1 to about 12% by weight. The oxides may be present as solid solutions or mixed oxides, or as mixture of oxides.

One or more of CaF₂, B₂O₃, Al₂O₃, MgO and K₂O may be included in the composition in addition to silicon, sodium, calcium and phosphorus oxides. The B₂O₃ may be present in an amount of about 0 to 10% by weight, the K₂O may be present in an amount of about 0 to about 8% by weight, the Al₂O₃ may be present in an amount of about 0 to about 4% by weight, the MgO may be present in an amount of about 0 to about 5% by weight and the CaF₂ may be present in an amount of about 0 to about 30% by weight.

One preferred glass is Bioglass® 45S5, which has a composition including about 45% by weight silicon dioxide (SiO₂), about 24.5% by weight sodium oxide (Na₂O), about 6% phosphorus oxide (P₂O₅), and about 24.5% by weight calcium oxide (CaO).

The bioactive glass of the bioactive glass compositions may be used in either particulate or extract form. Preferably, where particulate bioactive glass is used, particulate, non-interlinked bioactive glass is selected. This glass is in the form of small, discrete particles, rather than a fused matrix of particles or a mesh or fabric (woven or non-woven) of glass fibers. Under some conditions the discrete particles of the bioactive glass may tend to cling together because of electrostatic or other forces but these particles are still considered to be non-interlinked. Typically, the average particle size is about 90 microns or less. Preferably, the average particle size is less than about 20 microns, or, more preferably, less than about 5 microns, and even more preferably less than about 1 micron. Particle size, as used herein, is measured by SEM or other optical microscopy techniques, or by laser light scattering techniques (i.e., using a Coulter counter).

The bioactive glass may be prepared in any way known to those of skill in the art. For example, the bioactive glass may be provided as melt-derived glass, sol-gel derived glass or sintered glass particles. The sintered particles may be in sol-gel derived, or pre-reacted melt derived form. Melt derived glass typically is prepared by mixing grains of oxides or carbonates, melting and homogenizing the mixtures at high temperatures, generally about 1250 to about 1400° C. The molten glass can be fritted and milled to produce a small particulate material. Sol-gel derived glass is typically prepared by synthesizing an inorganic network by mixing metal alkoxides in solution, followed by hydrolysis, gelation, and low temperature (less than about 1000° C.) firing to produce glass.

The bioactive glass may also be used in extract form. An extract of bioactive glass is a solution of ions derived from bioactive glass. Typically, the solution of bioactive glass comprises ions and substantially no particles. By solution is included solutions, suspensions and dispersions of bioactive glass. For example, an extract of bioactive glass may be formed from a solution made by reacting bioactive glass particles in an appropriate solvent such as water or tris buffer for an appropriate amount of time to create a solution of bioactive glass. The solution may then be filtered and used as a bioactive glass extract which is substantially particle free. The ratio of ions in solution will depend on the bioactive glass starting material and the amount of time it reacts in solution. The ion ratios may be controlled by use of various bioactive glass materials or by varying the reaction time.

The bioactive glass or bioactive glass exact typically will be applied directly to hair in conjunction with a carrier. The carrier may be aqueous or nonaqueous. The carrier preferably will be aqueous, alcohol based or other organic carriers or combinations thereof. Alternatively, cosmetic compositions can be provided in the form of aerosol sprays, foams or gels. While the ratio of bioactive glass to carrier is not critical, the bioactive glass composition will typically be about 0.5 to about 20% of the total bioactive glass/carrier formulation, including any optional components. Preferably, the amount of bioactive glass in the total composition will be about 2 to about 10%.

The bioactive glass or bioactive glass extract composition generally will be provided in an effective hair-enhancing amount. An effective, hair-enhancing amount is an amount capable of providing a thin film or coating on the hair of bioactive glass or ions from the bioactive glass which are believed to interact with the keratin in the hair to provide beneficial qualities to the hair. Typically, these ions will be calcium, silicon and/or phosphorus. Other ions which may be present, depending on the composition of the bioactive glass, include sodium, magnesium, potassium, zinc, copper or silver, among others. The thin film typically will be about 0.1 to about 5 microns thick. Preferably, the thin film formed on the hair will be about 0.3 to about 2 microns thick.

Without being bound by any theory, it is believed that the application of bioactive glass or bioactive glass extract composition according to the methods herein causes a layer of hydroxyapatite or other calcium phosphate crystals to form on the hair surface. Moreover, the ions from the bioactive glass are believed to penetrate layers of the hair to form hydroxyapatite crystals within the layers of the hair and may penetrate layers of the hair to form chemical bonds bridging the layers of the cuticle.

The bioactive glass or bioactive glass extract composition generally will be applied to the hair for a time sufficient to treat the hair. Typically, this time will allow a coating or film to form or precipitate onto the hair. The ions in the coating may then interact with the hair to enhance the hair. In general, the time needed is about the time typically used to shampoo the hair, although a longer time may be used. The methods for treating hair of the invention may involve use of a hair treatment formulation which is removed or washed off after use, or a hair treatment formulation such as a gel, mousse, cream, lotion, air infused styling foam or spray formulation which is left on after application.

In one aspect of the invention, the bioactive glass or bioactive glass extract is applied to hair in a carrier in a rinse type of formulation. In other aspects of the invention, the bioactive glass or bioactive glass extract composition is applied to hair in a shampoo or conditioner formulation. Such formulations are well known in the art. Application of the bioactive glass composition to the hair is believed to build body in the hair, and make hair smooth and silky. The methods of the invention may be used to prevent or repair damage to the hair caused by mechanical, chemical or environmental factors.

The hair treatment of the present invention may also impart a beneficial anti-microbial or anti-fungal activity for the hair, scalp or skin. Such treatment may be useful against microbes such as B. subtilis, S. aureus, P. aureginosa, C. albicans, A. niger, E. coli, A. altemata, and C. xerosis. The hair treatment methods of the invention may also be used to improve curl retention, color receptivity, color stability, color retention, shine, and/or strength.

The hair treatment formulations of the present invention may further comprise one or more optional components known for use in shampoo or conditioning compositions, provided that the optional components are physically and chemically compatible with the bioactive glass composition described herein, or do not otherwise unduly impair product stability, aesthetics or performance. Concentrations of such optional components typically range from about 0.001% to about 10% by weight of the total composition.

Optional components include anti-static agents, dyes, organic solvents or diluents, pearlescent aids, foam boosters, surfactants or cosurfactants (nonionic, cationic, zwitterionic), pediculocides, pH adjusting agents, perfumes, preservatives, proteins, skin active agents, suspending agents, styling polymers, sunscreens, thickeners, vitamins, biotin, collagen, amino acids, protein hydrolyzates, herbals, penetration enhancers, permeation/binding agents, and viscosity adjusting agents. This list of optional components is not meant to be exclusive, and other optional components can be used.

The hair treatment methods of the invention preferably use compositions maintained at a pH appropriate for the particular application. Typically, the pH will be below about 10; preferably, the pH will be between about 3 and about 9.

The invention will now be more fully explained by the following examples. However, the scope of the invention is not intended to be limited to these examples.

### EXAMPLE

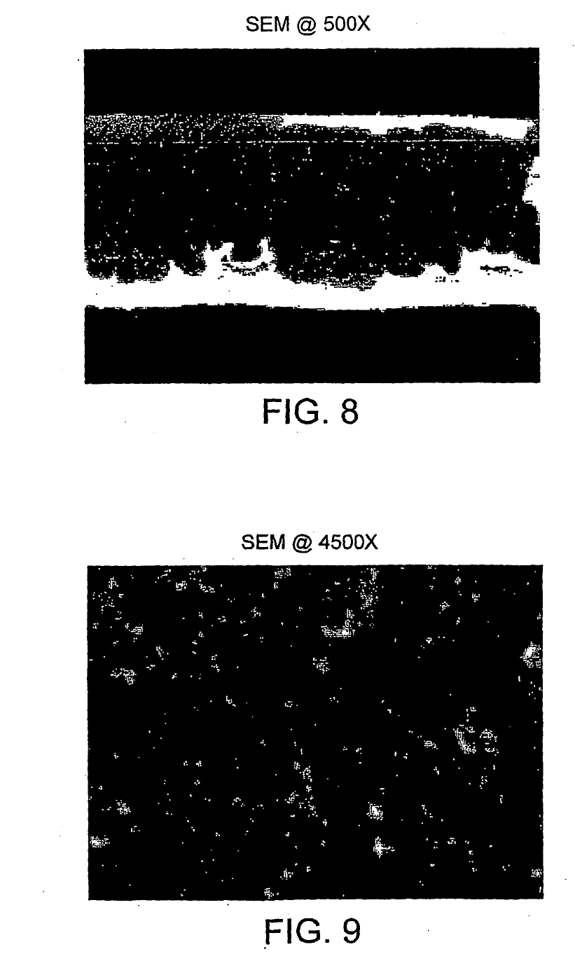

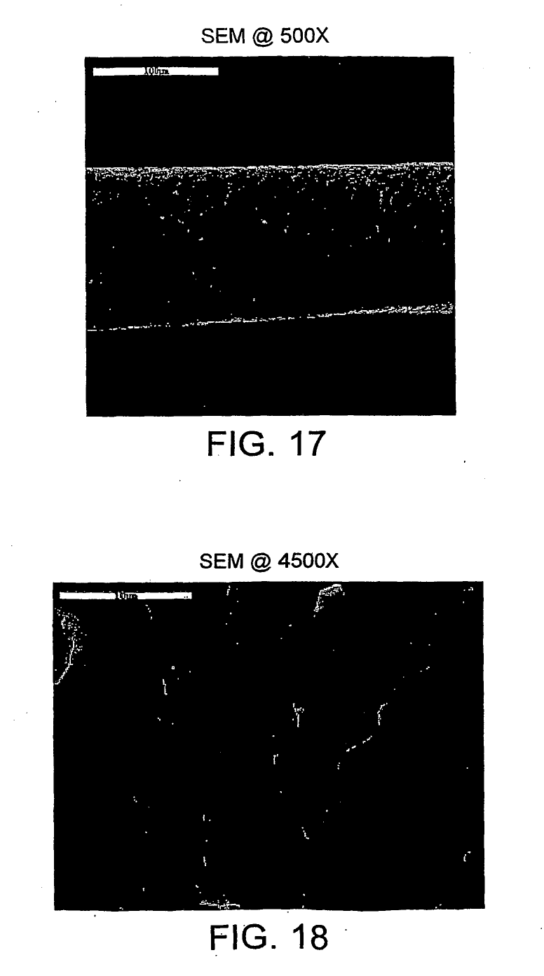

Four compositions were prepared and tested to evaluate the use of bioactive glass particulates, extracts and shampoo solutions. The bioactive glass used was Bioglass™ 45S5 powder with a particle size less than about 20 μm. These compositions are detailed in Table I. Two control formulations were also prepared and tested. The first control formulation corresponded with Groups #1 and #2 and contained only tris buffer. The second control formulation corresponded with Groups #3 and #4 and contained tris buffer and shampoo. These control formulations were viewed by SEM/EDS, with the results shown in FIGS. 8-10 and 17-19.

TABLE I

| | | | | Bioactive glass extract |
|---|---|---|---|---|
| | Tris Buffer | Shampoo | Bioactive | (200 ml of 15 wt % |
| | (200 ml) | (0.5 cc) | glass (0.3 g) | solution) |
| Group | ✓ | | ✓ | |
| #1 | | | | |
| Group | | | | ✓ |
| #2 | | | | |
| Group | ✓ | ✓ | ✓ | |
| #3 | | | | |
| Group | | ✓ | | ✓ |
| #4 | | | | |

Procedures:

Group #1—Tris Buffer and Bioactive Glass Particulate
1. 200 ml of tris buffer solution was delivered into a 500 ml flask and placed into a controlled temperature orbital shaker set at 37° C. and 200 RPM.
2. 0.3 grams of bioactive glass particulate with a particle size less than about 20 μm was placed into the preheated solution.
3. 0.25 grams of human hair was attached to a large piece of inert glass with a rubber band and placed into the tris buffer/bioactive glass suspension.
4. The sample was allowed to react for 20 hours and then was removed and thoroughly rinsed with high purity deionized water.
5. The sample was allowed to dry and mounted for SEM/EDS analysis or used for FTIR analysis.

Group #2—Bioactive Glass Extract
1. 90.0 grams of bioactive glass particulate with a particle size less than about 20 μm and 510.0 ml of tris buffer solution were allowed to vortex in a 1000 ml beaker for two hours at room temperature.
2. The solution was then filtered to form a 15.0 wt. % bioactive glass extract.
3. 0.25 grams of human hair was attached to a large piece of inert glass with a rubber band and placed into the bioactive glass extract solution.
4. The sample was allowed to react for 20 hours and then was removed and thoroughly rinsed with high purity deionized water.
5. The sample was allowed to dry and mounted for SEM/EDS analysis.

Group #3—Tris Buffer and Bioactive Glass Particulate with Shampoo

This procedure was identical to group # 1 with the addition of 0.5 cc of Suave Clarifying Shampoo with ammonium lauryl sulfate to the tris buffer/bioactive glass suspension.

Group #4—Bioactive Glass Extract with Shampoo

This procedure was identical to the group # 3 procedure with the addition of 0.5 cc of Suave Clarifying Shampoo with ammonium lauryl sulfate to the bioactive glass extract solution.

Results:

Group #1—Tris Buffer and Bioactive Glass Particulate

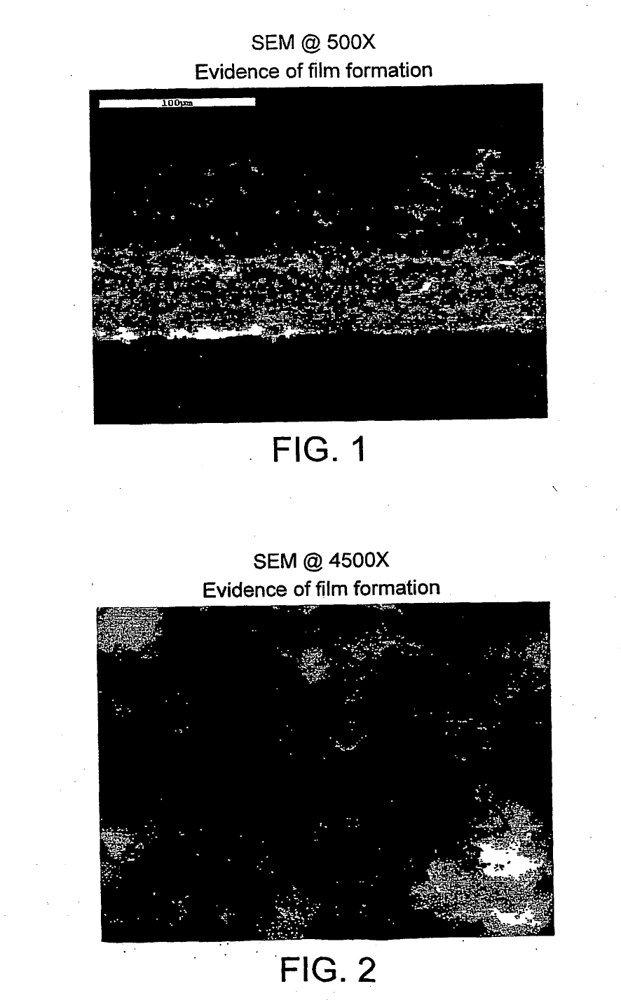

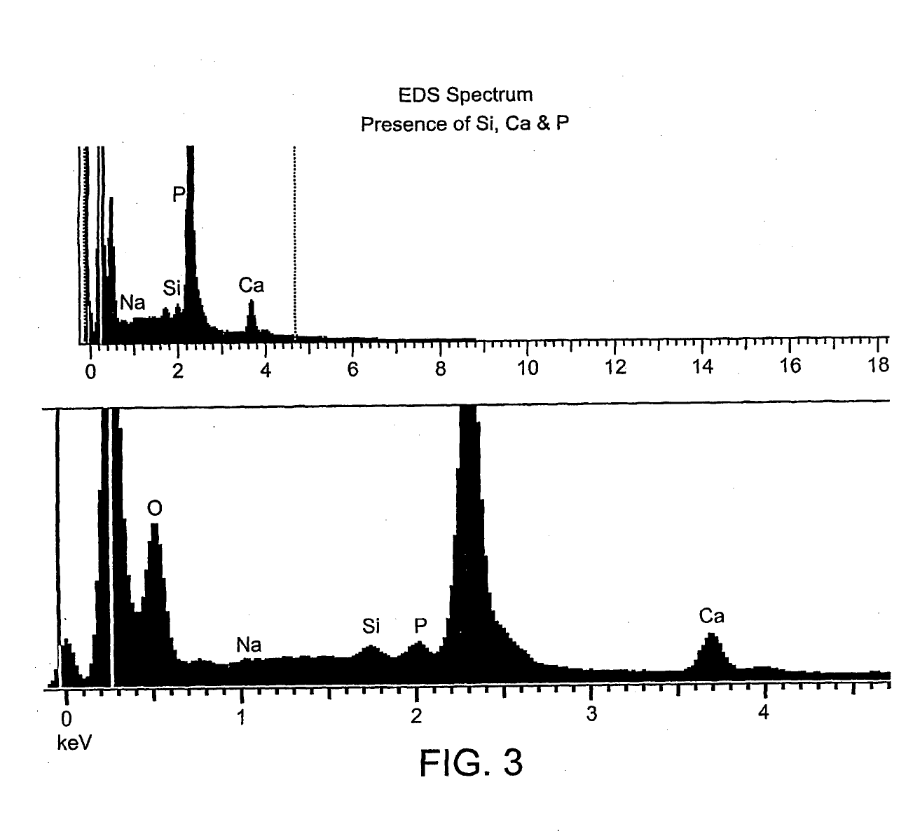

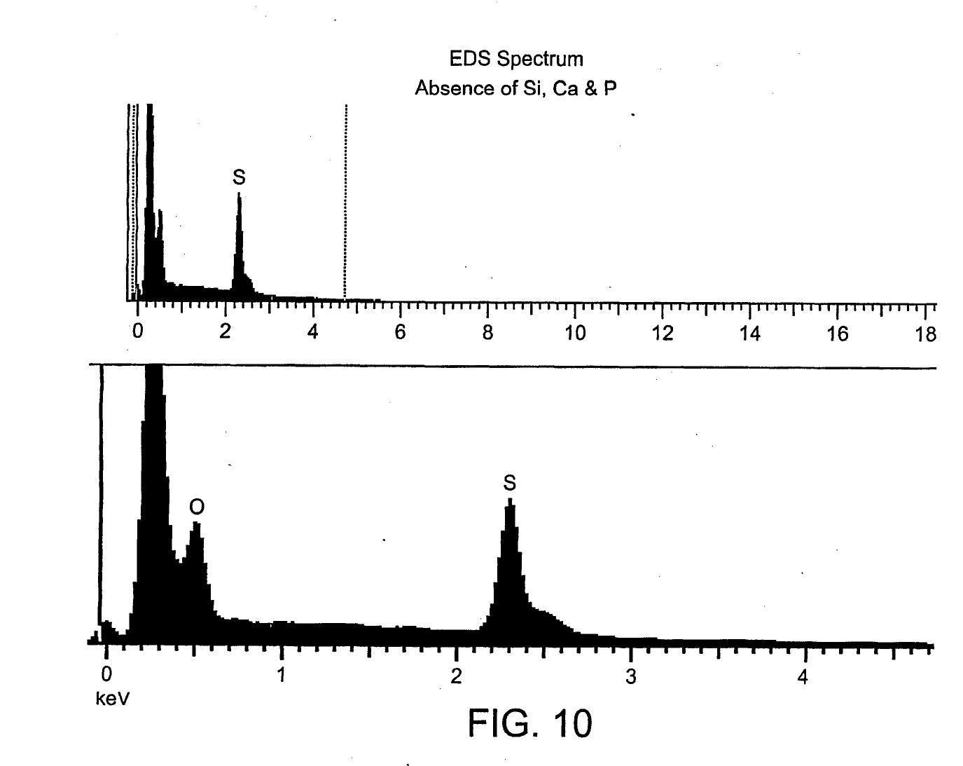

The SEM/EDS and FTIR results for group #1 are presented in FIGS. 1, 2, 3 and 4. The SEM micrographs indicate that a substantial film has been formed on the hair surface after reaction in tris buffer and bioactive glass particulate. Magnification to 4500× indicates that this film is non-uniform in coverage of the hair surface. The EDS analysis indicates the presence of Si, Ca, Na and P, which is not seen in the Group 1 and 2 control in FIG. 10. The presence of Si may indicate that some bioactive glass particulate may be present or that a soluble form of silicon has been incorporated on the hair surface.

Group #2—Bioactive Glass Extract

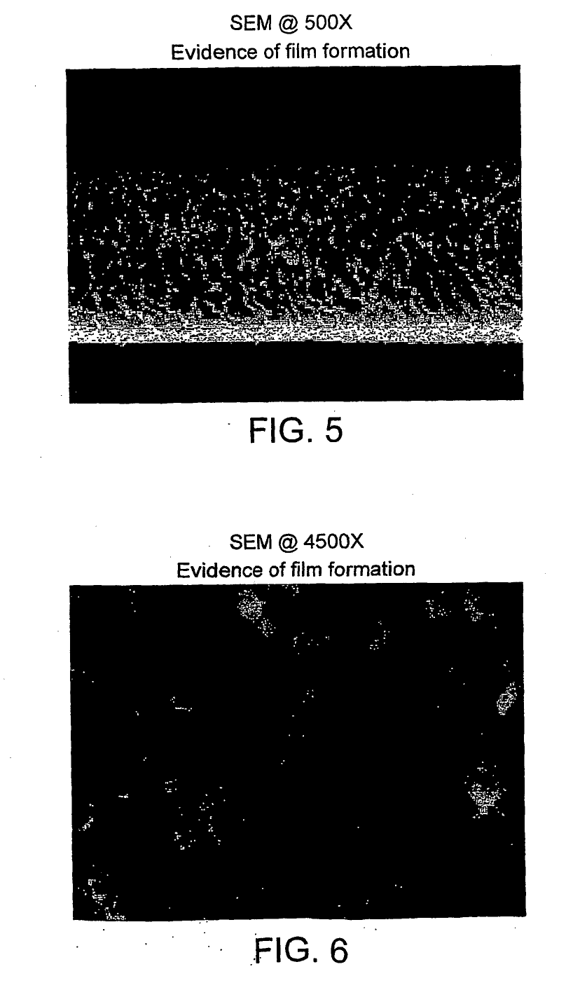

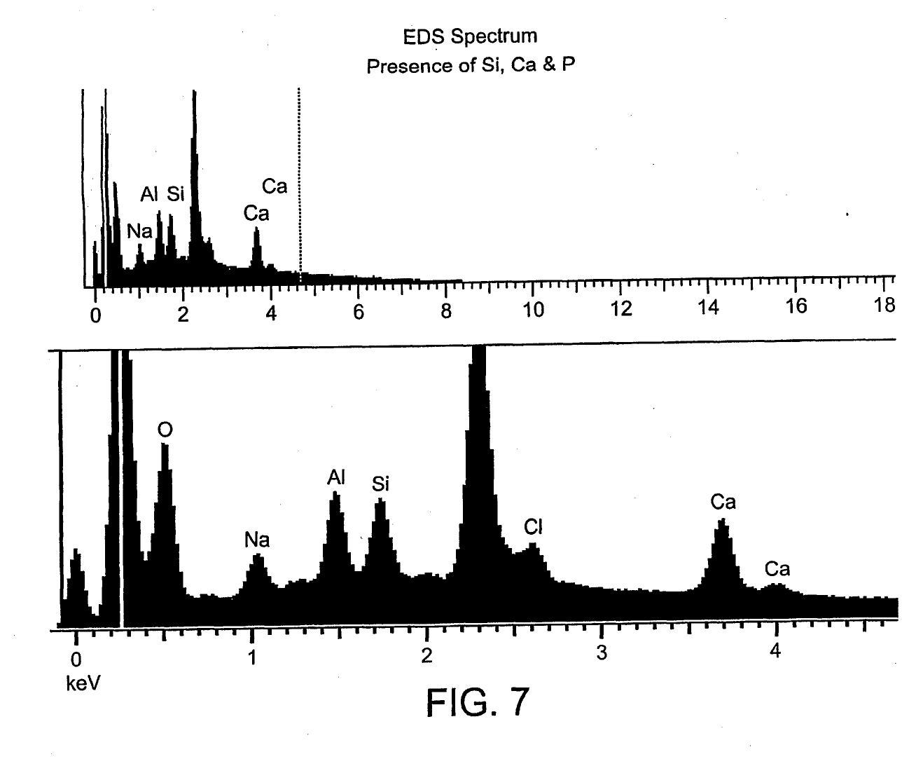

The SEM/EDS results for group #2 are presented in FIGS. 5, 6 and 7. The SEM micrographs indicate that a film has been formed on the hair surface after reaction in tris buffer and bioactive glass extract solution. Magnification to 4500× as shown in FIG. 6 indicates that this film is non-uniform in coverage of the hair surface. The film appears to be more dense on the edges of the cuticle scales of the hair structure. The EDS analysis of FIG. 7 indicates the presence of Si, Ca, Na and P, which is not seen in the Group 1 and 2 control in FIG. 10. The presence of Si may indicate that soluble form of silicon has been incorporated on the hair surface, as there are no bioactive glass particles in this filtered solution. The presence of Al is an artifact due to the aluminum mount used for SEM/EDS analysis.

Group #3—Tris Buffer and Bioactive Glass Particulate with Shampoo

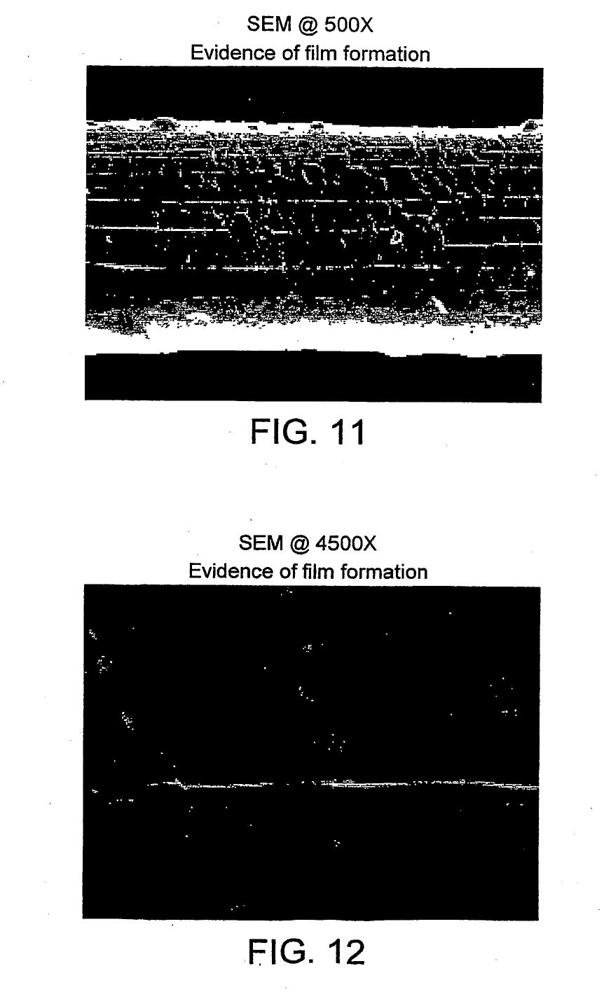

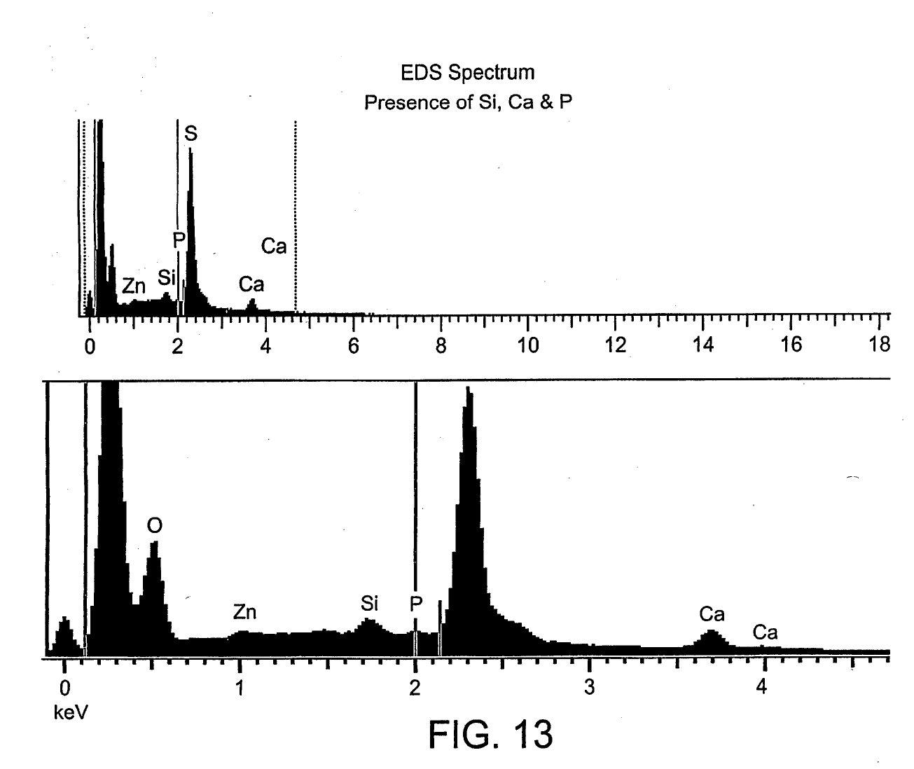

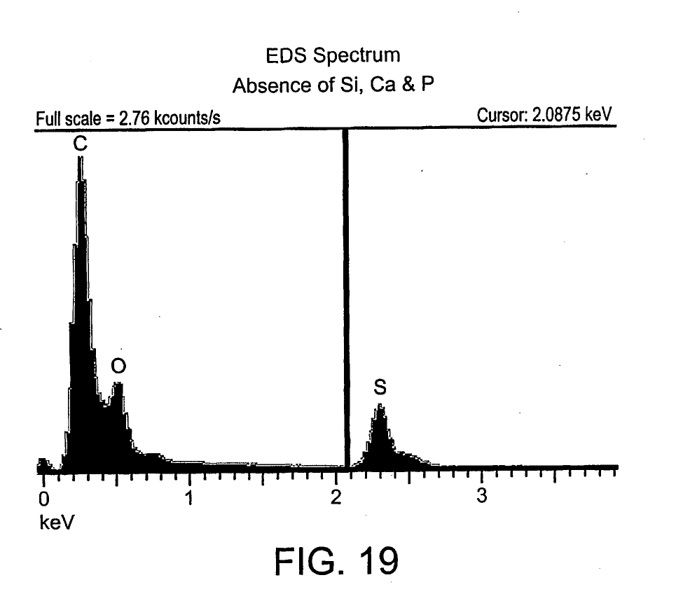

The SEM/EDS results for group #3 are presented in FIGS. 11, 12 and 13. The SEM micrographs indicate that a transparent film has been formed on the hair surface after reaction in the tris buffer, bioactive glass particulate and shampoo mixture. Magnification to 4500× in FIG. 12 indicates that this film is uniform in coverage of the hair surface. The linear cracks seen in the micrographs were formed during the SEM analysis. These cracks could be seen growing as the image was focused in the process of acquiring the micrograph. The EDS analysis indicates the presence of Si, Ca, Na and P, which is not seen in the Group 3 and 4 control in FIG. 19.

Group #4—Bioactive Glass Extract with Shampoo

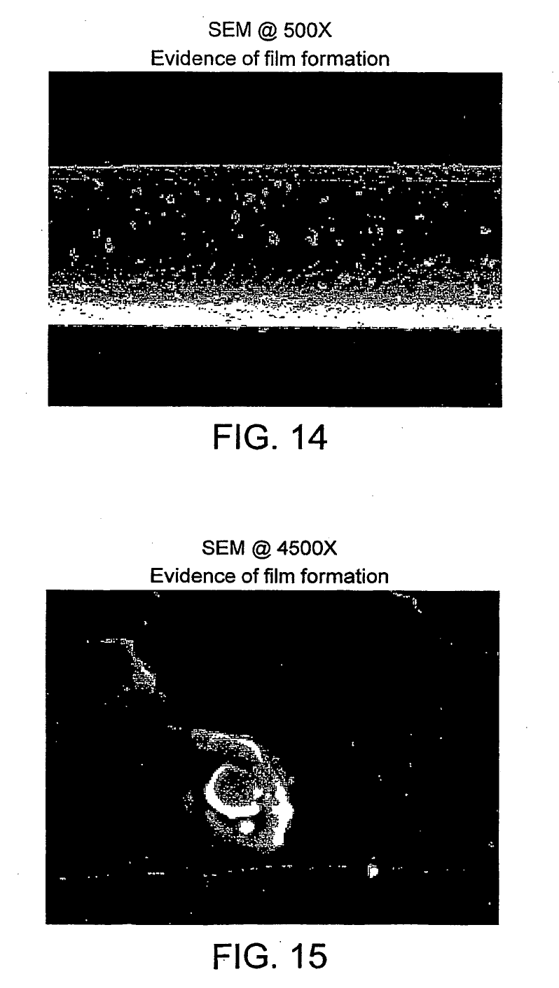

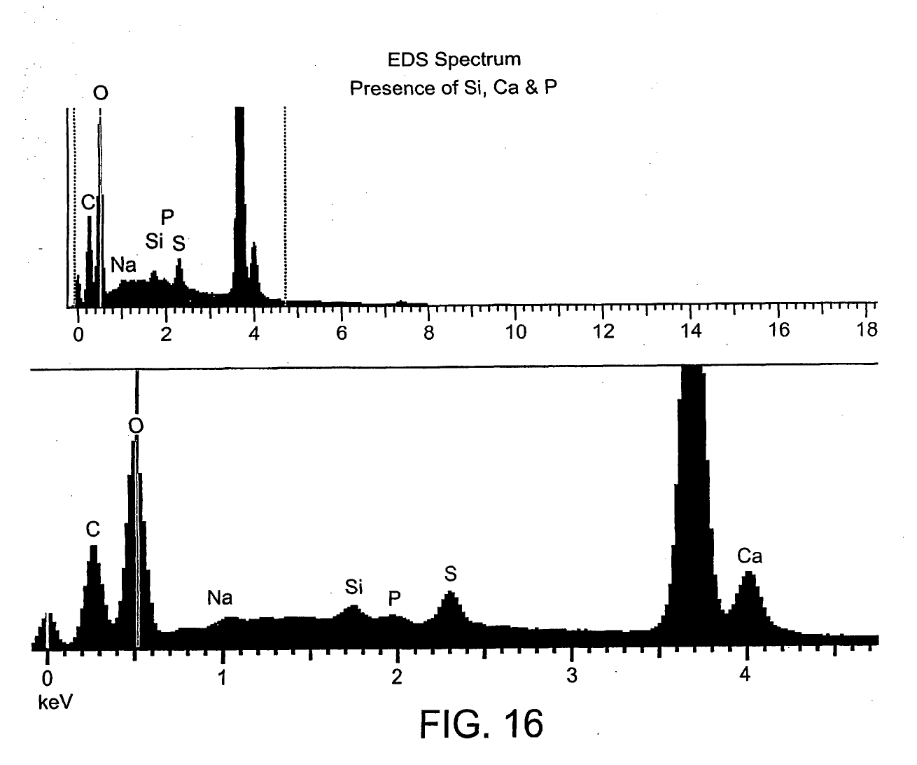

The SEM/EDS results for group #4 are presented in FIGS. 14, 15 and 16. The SEM micrographs indicate that a transparent film has been formed on the hair surface after reaction in the tris buffer, bioactive glass extract and shampoo mixture. Magnification to 4500× in FIG. 15 indicates that this film is uniform in coverage of the hair surface. The linear cracks seen in the micrographs were formed during the SEM analysis. These cracks could be seen growing as the image was focused in the process of acquiring the micrograph. The extent of the cracking was less for this group than seen for the group #3 sample. The EDS analysis indicates the presence of Si, Ca, Na and P, which is not seen in the Group 3 and 4 control in FIG. 19. The EDS analysis also indicates that this transparent film is thicker for this groups than the group #3 sample indicated by the significant reduction in the S peak at 2.30 keV and the significant increase in the alpha Ca peak at 3.75 keV. This increase in thickness could explain the reduction in cracking seen for this group as compared to the group #3 sample. The cause for this difference in thickness and dimensional stability of this transparent layer is unknown at this time.

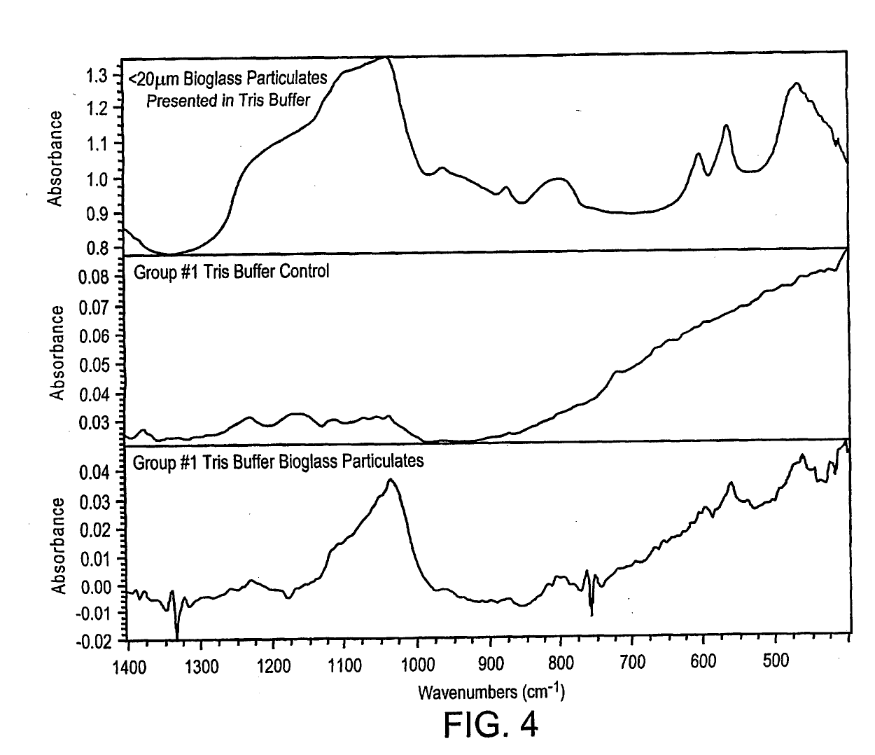

The data presented indicates that both bioactive glass suspensions and extracts in tris buffer (groups #1 and #2) have the ability to deliver calcium, phosphorus and silicon to the hair surface. The SEM/EDS analysis indicates that a non-continuous film is being formed on the surface of the hair and is thicker on the edges of the cuticle scales of the hair structure. The FTIR analysis from group #1 shown in FIG. 4 indicates that the calcium and phosphorus are in the form of crystalline hydroxyapatite. The data also indicates that the morphology of this film changes with the addition of a commercial shampoo. The resulting film is continuous and transparent in nature, as seen with Groups #3 and #4. The EDS analysis indicates that this film contains calcium, phosphorus and silicon and is thicker in the extract solution Group #4 than the suspension group #3 mixed with shampoo. Thus, the data shows that bioactive glass interacts with human hair.

While the invention has been described with preferred embodiments, it is to be understood that variations and modifications may be resorted to as will be apparent to thee skilled in the art.

1. A method for treating hair comprising applying a bioactive glass composition in an effective hair-enhancing amount to the hair for a sufficient time to form a coating on the hair comprising silicon, calcium and/or phosphorus ions.

2. The method of claim 1 wherein the coating on the hair further comprises sodium ions.

3. The method of claim 1 wherein the bioactive glass composition comprises a carrier and non-interlinked particles of bioactive glass comprising about 40 to about 86% by weight of SiO₂, about 4 to about 46% by weight CaO and about 1 to about 15% by weight P₂0₅.

4. The method of claim 3 wherein the bioactive glass composition has a pH below about 10.

5. The method of claim 3 wherein the bioactive glass composition has a pH between about 3 and about 9.

6. The method of claim 4 wherein the non-interlinked particles have a particle size less than about 90 microns.

7. The method of claim 4 wherein the non-interlinked particles have a particle size less than about 20 microns.

8. The method of claim 4 wherein the non-interlinked particles have a particle size less than about 5 microns.

9. The method of claim 1 wherein the bioactive glass composition comprises non-interlinked particles of bioactive glass comprising about 40 to about 68% by weight of SiO₂, about 10 to about 35% by weight CaO, about 1 to about 12% by weight P₂O₅ and about 5 to about 30% by weight Na₂O.

10. The method of claim 1 wherein the coating is about 0.1 to about 5 microns thick.

11. The method of claim 1 wherein the bioactive glass composition comprises bioactive glass extract and a carrier.

12. The method of claim 1 wherein the hair is human hair.

13. The method of claim 1 wherein the hair is damaged hair.

14. A method for treating hair comprising applying to the hair a hair treatment formulation comprising a carrier and an effective hair-enhancing amount of a bioactive glass composition comprising either non-interlinked particles of bioactive glass or a bioactive glass extract.

15. The method of claim 14 wherein the bioactive glass composition comprises non-interlinked particles of bioactive glass comprising about 40 to about 86% by weight of SiO₂, about 4 to about 46% by weight CaO and about 1 to about 15% by weight P₂O₅.

16. The method of claim 14 wherein the bioactive glass composition comprises bioactive glass extract comprising a solution of bioactive glass comprising about 40 to about 86% by weight of SiO₂, about 4 to about 46% by weight CaO and about 1 to about 15% by weight P₂O₅.

17. The method of claim 14 wherein the hair treatment formulation is a shampoo or conditioner and the method further comprises rinsing the shampoo or conditioner out of the hair.

18. The method of claim 14 wherein the hair treatment formulation is a gel, mousse, cream, lotion, air infused styling foam or spray composition and the method further comprises leaving the hair treatment formulation on the hair.

19. The method of claim 14 wherein the hair treatment formulation is applied to damaged hair.

20. The method of claim 14 wherein the hair treatment formulation further comprises anti-static agents, dyes, organic solvents or diluents, pearlescent aids, foam boosters, surfactants or cosurfactants, pediculocides, pH adjusting agents, perfumes, preservatives, proteins, skin active agents, suspending agents, styling polymers, sunscreens, thickeners, vitamins, biotin, collagen, amino acids, protein hydrolyzates, herbals, penetration enhancers, permeation/binding agents, or viscosity adjusting agents.

21. The method of claim 20 wherein the bioactive glass composition is present in an amount of about 2 to about 10% of the total composition.

22. The method of claim 21 wherein the pH is between about 3 and about 9.

23. A method for treating hair comprising applying a composition comprising an effective, hair-enhancing amount of bioactive glass particles to the hair surface for a sufficient amount of time to provide that a layer of hydroxyapatite or other calcium phosphate crystals are formed on the hair surface and ions from the bioactive glass penetrate layers of the hair to form hydroxyapatite crystals within the layers of the hair.

24. The method of claim 23, wherein the composition comprising the bioactive glass particles further comprises an aqueous carrier and is applied to hair damaged by mechanical, chemical or environmental factors.

25. The method of claim 23, wherein the composition comprising the bioactive glass particles further comprises an aqueous carrier and is applied to hair to prevent damage by mechanical, chemical or environmental factors.

26. The method of claim 23 wherein the composition is applied to the hair to improve curl retention, color receptivity, color stability, color retention, shine, and/or strength.

27. The method of claim 23, wherein the composition further comprises biotin, collagen, amino acids, proteins, protein hydrolyzates, vitamins, herbals, penetration enhancers, permeation/binding agents, dyes or fragrances.

28. The method of claim 23, wherein the average particle size of the bioactive glass particles is less than about 20 microns.

29. The method of claim 23, wherein the average particle size of the bioactive glass particles is less than about 5 microns.

30. The method of claim 29, wherein the average particle size of the bioactive glass particles is less than about 1 micron.

31. The method of claim 23, wherein the composition comprising bioactive glass particles comprises non-interlinked particles of bioactive glass comprising about 40 to about 68% by weight of SiO₂, about 10 to about 35% by weight CaO, about 1 to about 12% by weight P₂O₅ and about 5 to about 30% by weight Na₂O.

32. The composition of claim 27 wherein the bioactive glass particles are present in the composition in an amount of about 0.5 to about 20 percent by weight of said composition.

33. The method of claim 23, wherein the hair is human hair.

34. The method of claim 23 wherein the hair is dog or cat hair.
